(19) United States
(12) Patent Application Publication
(10) Pub. No.: US 2024/0299588 A1
(43) Pub. Date: Sep. 12, 2024

(54) NERVE LABEL

(71) Applicant: UCL Business Ltd, London Greater London (GB)

(72) Inventors: Elnaz YAGHINI, London Greater London (GB); Alexander John MACROBERT, London Greater London (GB); James PHILLIPS, London Greater London (GB)

(73) Assignee: UCL Business Ltd, University College London, London Greater London (GB)

(21) Appl. No.: 18/579,784

(22) PCT Filed: Jul. 18, 2022
(86) PCT No.: PCT/GB2022/051847

§ 371 Date: Jan. 16, 2024

(30) Foreign Application Priority Data
Jul. 16, 2021 (GB) 2110249.6

(51) Int. Cl. A61K 49/00; A61B 5/00

(52) U.S. Cl. CPC A61K 49/0021; A61B 5/0071; A61B 5/4893; A61B 2505/05

(57) ABSTRACT

The invention relates to a method of visualising a peripheral nerve, the method comprising contacting the nerve with a 5-aminolevulinate or a derivative thereof.

### CROSS REFERENCE TO RELATED APPLICATIONS

This patent application is a U.S. National Stage Application of PCT Patent Application No. PCT/GB2022/051847, filed on Jul. 18, 2022, which claims the benefit and priority of GB Patent Application No. 2110249.6, filed on Jul. 16, 2021, the contents of both of which are incorporated by reference herein in their entirety.

### FIELD OF THE INVENTION

The invention relates to a method of visualising a peripheral nerve. The invention also relates to a method of fluorescently labelling a peripheral nerve, and medical uses of a 5-aminolevulinate or a derivative thereof.

### BACKGROUND

Preservation of the functional and structural properties of nerves and blood vessels during surgery is imperative for clinical benefit, and rapid recovery, together with reduced post-operative morbidities and increased patient satisfaction post-surgery. Unintended nerve damage and transection during surgery is one of the leading complications (major morbidity) in both open and minimally invasive surgery—such as laparoscopic and robotic surgery. This problem is associated with many surgeries (both cancer and non-cancer surgeries) and is particularly common in head and neck surgery, prostatectomy, thoracic and cardiac surgeries, trauma and orthopaedic surgeries, colorectal and gynaecological surgeries.

Currently, during an operation, a surgeon identifies peripheral nerves based on their appearance through visual inspection and the anatomical location of various organs and tissues within the body. However, these techniques are not always reliable because each patient is different and certain anatomical differences can cause issues during surgery together with the inherent difficulties associated with seeing and distinguishing thin and buried nerves.

Complications arising from nerve injuries substantially reduce a patient's quality of life leading to life-long complications and significant morbidity including chronic pain, muscle paralysis, sensory loss, numbness, loss of function of limbs and organs. For instance, in urologic surgery, urinary incontinence has been reported in up to 20% of patients following prostatectomy.

The aim of the invention is to overcome the problems associated with the techniques currently used to identify nerves during surgery.

### STATEMENTS OF THE INVENTION

5-aminolevulinic acid (5-ALA) is a naturally occurring amino acid that is present in most nucleated cells of the body. It is an early intermediate in the haem biosynthetic pathway and is metabolised into the fluorescent porphyrin, protoporphyrin IX (PpIX), before ultimately being converted into haem. Exogenous administration of 5-ALA leads to transient accumulation of PpIX in most cells, particularly rapidly dividing cells with high metabolic activity (e.g. cancer cells). Consequently, 5-ALA has been used clinically for over 20 years to detect high-grade glioma tumours by detecting (increased) fluorescence typically 3 to 4 hours after 5-ALA has been administered (fluorescence emitted by cancer cells returns to its basal levels by 24 hours after administration).

The inventors have unexpectedly found that peripheral nerves selectively retain high levels of fluorescence compared to background fluorescence levels up to 7 days after administration of 5-ALA. This occurs because PpIX is cleared from peripheral nerves at a significantly slower rate compared to that of surrounding tissue, e.g. fat, muscle etc (see Example 1). The inventors also believe but do not wish to be bound by the hypothesis that peripheral nerves selectively retain high levels of fluorescence because the enzyme (i.e. ferrochelatase) responsible for converting PpIX into the non-fluorescent molecule, haem, may be present at low concentrations in peripheral nerves where PpIX is located.

Thus, according to a first aspect of the invention, there is provided a method of visualising a peripheral nerve, the method comprising:
contacting the nerve with a 5-aminolevulinate or a derivative thereof;
exposing the nerve to light at least about 1 hour after contact with the 5-aminolevulinate or a derivative thereof in order to cause the peripheral nerve to emit fluorescence; and
detecting the fluorescence emitted by the nerve in order to visualise the peripheral nerve.

In another aspect of the invention, there is provided a method of visualising a peripheral nerve, the method comprising:
exposing a peripheral nerve, which has been contacted with a 5-aminolevulinate or a derivative thereof, to light at least about 1 hour after contact with the 5-aminolevulinate or derivative thereof, in order to cause the peripheral nerve to emit fluorescence; and
detecting the fluorescence emitted by the nerve in order to visualise the peripheral nerve.

According to a second aspect of the invention, there is provided a method of fluorescently labelling a peripheral nerve, the method comprising contacting the peripheral nerve with a 5-aminolevulinate or a derivative thereof at least about 1 hour prior to exposing the nerve to light, thereby fluorescently labelling the peripheral nerve.

Preferably, the method comprises selectively labelling a peripheral nerve. Preferably, the method comprises labelling a peripheral nerve in a subject.

According to a third aspect, there is provided a method of performing surgery on a subject, the method comprising:
administering a 5-aminolevulinate or a derivative thereof to a subject;
exposing a peripheral nerve of the subject to light at least about 1 hour after administration of the 5-aminolevulinate or derivative thereof, in order to cause the peripheral nerve to emit fluorescence;
detecting the fluorescence emitted by the peripheral nerve of the subject in order to visualise the peripheral nerve; and
performing surgery on the subject while the peripheral nerve is being visualised.

In another aspect, there is provided a method of performing surgery on a subject, the method comprising:
exposing a peripheral nerve of a subject, to whom a 5-aminolevulinate or a derivative thereof has been administered, to light at least about 1 hour after the 5-aminolevulinate or a derivative thereof administration, in order to cause the peripheral nerve to emit fluorescence;
detecting the fluorescence emitted by the peripheral nerve of the subject in order to visualise the peripheral nerve; and
performing surgery on the subject while the peripheral nerves is being visualised.

According to a fourth aspect, there is provided a 5-aminolevulinate or a derivative thereof for use in (a method of) surgery on a subject.

The (method of) surgery may comprise:
administering the 5-aminolevulinate or derivative thereof to the subject;
exposing a peripheral nerve of the subject to light at least about 1 hour after administration of the 5-aminolevulinate or derivative thereof, in order to cause the peripheral nerve to emit fluorescence;
detecting the fluorescence emitted by the peripheral nerve of the subject in order to visualise the peripheral nerve; and
performing surgery on the subject while the peripheral nerve is being visualised.

In another aspect, there is provided a 5-aminolevulinate or a derivative thereof for use in (a method of) surgery on a subject, the (method of) surgery comprising:
exposing a peripheral nerve of a subject, to whom a 5-aminolevulinate or a derivative thereof has been administered, to light at least about 1 hour after the administration, in order to cause the peripheral nerve to emit fluorescence;
detecting the fluorescence emitted by the peripheral nerve of the subject in order to visualise the peripheral nerve; and
performing surgery on the subject while the peripheral nerve is being visualised.

The surgery may be one or more selected from the group comprising: cancer surgery, non-cancer surgery, head and neck surgery, mastectomy, thoracic surgery, prostatectomy, cardiothoracic surgery (e.g. coronary artery bypass), gynaecological surgery, colorectal surgery, and trauma and orthopaedic surgery. The surgery may comprise identifying a severed peripheral nerve.

In another aspect of the invention, there is provided a method of treating a disease or disorder in a subject, the method comprising:
visualising a peripheral nerve using a method according to the invention; or
fluorescently labelling a peripheral nerve using a method according to the invention.

In another aspect of the invention, there is provided a 5-aminolevulinate or a derivative thereof for use in (a method of) treating a disease or disorder in a subject.

The method may comprise:
visualising a peripheral nerve using a method according to the invention; or
fluorescently labelling a peripheral nerve using a method according to the invention.

In one embodiment, the invention may further comprise administering a therapeutic agent to the subject in order to treat the disease or disorder.

In another aspect of the invention, there is provided a therapeutic agent for use in (a method of) treating a disease or disorder in a subject, the method comprising:
visualising a peripheral nerve using a method according to the invention; or
fluorescently labelling a peripheral nerve using a method according to the invention.

The disease or disorder may be one or more diseases/disorders selected from the group comprising: cancer, a cardiovascular disease/disorder (e.g., hypertension), a pulmonary disease/disorder, a gynaecological disease/disorder, a colorectal disease/disorder, ocular disease/disorder, a hepatic disease/disorder, a kidney disease/disorder, an autoimmune disease/disorder, a neural disease/disorder, muscular disease/disorder, and a bone disease/disorder.

Peripheral nerves, like all tissues, emit background levels of fluorescence (autofluorescence) in response to exposure to light. The invention described herein is advantageous because it results in the intensity of fluorescence emitted by peripheral nerves being selectively increased (i.e. compared to background fluorescence intensity) and retained for a prolonged period of time (e.g. from about 1 hour to about 7 days) after administration of the 5-aminolevulinate (e.g. 5-ALA) or a derivative thereof to a subject. Thus, medical practitioners (e.g. surgeons) can visualise nerves in real-time during surgery. Consequently, the risk of unintentional damage to peripheral nerves during surgery is significantly reduced.

The step of administering a 5-aminolevulinate (e.g. 5-ALA) or a derivative thereof to a subject may comprise topical or systemic administration of 5-aminolevulinate (e.g. 5-ALA) or a derivative thereof. Systemic administration may be oral and/or intravenous administration. Preferably the 5-aminolevulinate (e.g. 5-ALA) or derivative thereof is administered orally. Oral administration of a 5-aminolevulinate is well tolerated.

The step of exposing the peripheral nerve to light may comprise exposing the peripheral nerve to light of a specific wavelength. The wavelength may be about 600 nm to about 700 nm (red light) or about 380 nm to about 495 nm (blue light). The peripheral nerve may be exposed to red light because red light penetrates biological tissue more deeply than shorter wavelengths (e.g. blue) and thus enables visualisation of nerves located deeper within a tissue or a body. The peripheral nerve may be exposed to light of a wavelength of about 620 nm to about 650 nm in order to provide optimum contrast. The peripheral nerve may be exposed to blue light because it is more effective at visualising superficial nerves. Preferably the peripheral nerve is exposed to (or excited with) blue light, e.g. 405 nm and fluorescence is recorded at wavelengths in the red light part of the spectrum (e.g. 620-650 nm).

The “exposing” step may not comprise surgery, such as cutting tissue surrounding a peripheral nerve so that the peripheral nerve per se is in contact with the ambient environment. The skilled person will appreciate that the “exposing” step may comprise illuminating the subject or a body part of the subject that comprises the peripheral nerve. Thus, the light may pass through biological tissue of the subject in order to contact the peripheral nerve. Therefore, the exposing step may comprise indirectly exposing the peripheral nerve to light. However, if, for example, the peripheral nerve is in vitro or has been surgically revealed (so that it is in contact with ambient environment), the light may not need to pass through biological tissue to contact the peripheral nerve.

It is generally preferred that the peripheral nerve is exposed to the light for the minimum amount of time needed for PpIX within the nerve(s) to emit fluorescence.

Accordingly, in vitro or ex vivo, the peripheral nerve may be exposed to light for a duration of about 30 seconds or less, about 25 seconds or less, about 20 seconds or less, about 15 seconds or less, about 10 seconds or less, about 9 seconds or less, about 8 seconds or less, about 7 seconds or less, about 6 seconds or less, about 5 seconds or less, about 4 seconds or less, about 3 seconds or less, about 2 seconds or less, about 1 second or less, about 0.9 seconds or less, about 0.8 seconds or less, about 0.7 seconds or less, about 0.5 seconds or less, about 0.4 seconds or less, about 0.3 seconds or less, about 0.2 seconds or less, or about 0.1 seconds or less. Preferably, the peripheral nerve is exposed to light for a duration of about 2 seconds or less, or I second or less.

The skilled person will appreciate that, when exposing a nerve to light in vivo or ex vivo, the length of the exposure time may depend on the location and depth of the peripheral nerve within the subject as well as the size (diameter and/or length) of the nerve. Thus, while clear fluorescence images can be obtained by using a very short exposure time (i.e. less than a second), a surgeon may need to inspect the surgical field for a longer period of time to visualise all the nerves in the field. Accordingly, in vivo or ex vivo, the peripheral nerve may be exposed to light for a duration of about 1 minute or more, about 2 minutes or more, about 5 minutes or more, about 10 minutes or more, about 20 minutes or more about 30 minutes or more, about 45 minutes or more, about 1 hour or more, about 2 hours or more, about 3 hours or more, about 4 hours or more, about 5 hours or more, about 6 hours or more, about 7 hours or more, about 8 hours or more, about 9 hours or more, about 10 hours or more, about 12 hours or more, about 14 hours or more, about 16 hours or more, about 18 hours or more, about 20 hours or more, about 24 hours or more, about 30 hours or more, about 36 hours or more, about 42 hours or more. Thus, the peripheral nerve may be exposed to light for about 1 minute to about 42 hours. Preferably, the peripheral nerve is exposed to light for a duration of 20 minutes to about 2 hours, or 30 minutes to about 1 hour.

The skilled person will appreciate that the purpose of the contacting/administering step is to enable the 5-aminolevulinate or derivative thereof to enter into the peripheral nerve(s), so that it can be metabolised into PpIX. The time-point at which fluorescence is detected after exogenous administration of the 5-aminolevulinate or derivative thereof (or after contact with a nerve) will be determined by the magnitude of the signal to background ratio (SBR), the ‘signal’ being the fluorescence emitted by peripheral nerves, which includes fluorescence emitted by PpIX created by metabolism of the exogenous 5-aminolevulinate or derivative thereof, and the ‘background’ being autofluorescence. The larger the SBR, the easier it is to identify and/or visualise the peripheral nerve(s) during surgical procedures. This enables one to visualise peripheral nerves in real-time during intraoperative procedures. The inventors have found that peripheral nerves can be visualised as short as 1 hour after the contacting step or administration of a 5-aminolevulinate (e.g. 5-ALA) or a derivative thereof. However, the highest SBR may be achieved at about 24 hours after administration or contacting the nerve with the 5-aminolevulinate or a derivative thereof. At about 24 hours after contact or administration, PpIX has effectively been cleared from all adjacent tissues while still being present in the peripheral nerves.

Thus, the invention may comprise exposing a peripheral nerve to light at about 1 to about 7 days after the contacting or administering step. The invention may comprise exposing a peripheral nerve to light at least about 1 hour, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 28 hours, at least about 32 hours, at least about 36 hours, at least about 2 days, at least about 3 days, at least about 4 days, at least about 5 days, at least about 6 days, or up to about 7 days after the contacting step or administration of the 5-aminolevulinate (e.g. 5-aminolevulinate) or a derivative thereof. Preferably, the invention comprises exposing a peripheral nerve to light at about 24 hours or more, preferably at about 24 hours, after contacting the nerve with the 5-aminolevulinate (e.g. 5-ALA) or a derivative thereof (or administration of the 5-aminolevulinate or a derivative thereof).

The invention may comprise exposing a peripheral nerve to light about 1 hour to about 7 days, about 4 hours to about 6 days, about 8 hours to about 5 days, about 12 hours to about 4 days, about 4 hours to about 2 days, about 8 hours to about 36 hours, about 12 hours to about 36 hours, about 8 hours to about 30 hours, about 12 hours to about 30 hours, or about 20 hours to about 30 hours after contacting the nerve with the 5-aminolevulinate or a derivative thereof (or administration of the 5-aminolevulinate or a derivative thereof). Preferably, the invention comprises exposing a peripheral nerve to light about 20 hours to about 30 hours after the contacting the nerve with a 5-aminolevulinate or a derivative thereof (or administration of the 5-aminolevulinate or a derivative thereof).

The invention may comprise contacting the peripheral nerve with a 5-aminolevulinate (e.g. 5-ALA) or a derivative thereof at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 28 hours, at least about 32 hours, at least about 36 hours, at least about 2 days, at least about 3 days, at least about 4 days, at least about 5 days, at least about 6 days, or up to about 7 days prior to exposing the nerve to light.

The invention may comprise contacting the peripheral nerve with a 5-aminolevulinate (e.g. 5-ALA) or a derivative thereof about 1 hour to about 7 days prior to exposing the nerve to light, about 4 hours to about 4 days prior to exposing the nerve to light, about 8 hours to about 3 days prior to exposing the nerve to light, about 12 hours to about 2 days hours prior to exposing the nerve to light, about 4 hours to about 36 hours prior to exposing the nerve to light, about 8 hours to about 36 hours prior to exposing the nerve to light, about 12 hours to about 36 hours prior to exposing the nerve to light, about 8 hours to about 30 hours prior to exposing the nerve to light, about 12 hours to about 30 hours prior to exposing the nerve to light, or about 20 hours to about 30 hours prior to exposing the nerve to light. Preferably, the invention comprises administering or contacting the peripheral nerve with a 5-aminolevulinate (e.g. 5-ALA) or a derivative thereof about 20 hours to about 30 hours prior to exposing the nerve to light.

The step of detecting fluorescence emitted by the peripheral nerve may comprise using a fluorescence imaging technique/device known in the art, such as a selection of one or more of an operating microscope or a laparoscope, including robotic imaging. Fluorescence imaging is used because it provides real-time and high resolution images, it is highly compatible with the intraoperative setting, it can easily be integrated into surgical routines, and it is relatively inexpensive. The step of detecting fluorescence emitted by the peripheral nerves may additionally or alternatively refer to viewing fluorescence emitted by a peripheral nerve with one eyes or recording it (e.g. as a photograph or a film).

The step of detecting fluorescent light may involve (selectively or specifically) detecting the fluorescence emitted from PpIX in peripheral nerves. This is because exogenously administered 5-aminolevulinate (e.g. 5-ALA) is metabolised into PpIX once it enters into most cells, including peripheral nerves. However, the inventors believe (but do not wish to be bound by the theory that) unlike in most tissues, PpIX selectively accumulates in peripheral nerves for a longer period of time, e.g. up to about 7 days after 5-aminolevulinate (e.g. 5-ALA) has been administered possibly because the enzyme responsible for converting PpIX into haem (i.e. ferrochelatase) is present in low concentrations in peripheral nerves where the PpIX is located compared to the concentration in other surrounding tissues. Consequently, the PpIX clearance rate is much faster in other surrounding tissues compared to the clearance rate in peripheral nerves.

The step of detecting fluorescence may comprise detecting fluorescence emitted at about 600 nm to about 750 nm (red/near-infrared fluorescence). Preferably, the light is detected at the fluorescence emission peaks of PpIX. Thus, the step of detecting fluorescence may comprise detecting fluorescence emitted at about 635 nm and/or 710 nm. The step of detecting fluorescence may comprise detecting fluorescence emitted at about 600 nm to about 750 nm, about 620 nm to about 650 nm, or about 635 nm after the nerve has been exposed to blue light e.g. light at about 405 nm. The step of detecting fluorescence may comprise detecting fluorescence emitted at about 650 nm to about 750 nm, or about 710 nm after the nerve has been exposed to light of a wavelength of about 635 nm.

The SBR may be calculated by comparing the intensity of fluorescence emitted by a peripheral nerve (after contact with a 5-aminolevulinate or a derivative thereof) to the intensity of background fluorescence. Background fluorescence may be autofluorescence emitted by surrounding structures or tissue, such as bones, adipose (fat) and/or muscle. For intraoperative surgery the nerve fluorescence SBR may be calculated based on surrounding tissues, such as muscle. The skilled person will appreciate that the SBR will vary based on the nerve location and its size. Nevertheless, the SBR may be at least about 4, at least about 10, at least about 15, or at least about 20. The SBR may be at least about 4, at least about 10, at least about 15, or at least about 20 compared to muscle. The SBR may be at least about 4, at least about 5, at least about 10, at least about 15, or at least about 20 compared to adipose. Preferably, the SBR is about 4-5.

According to a fifth aspect, there is provided a 5-aminolevulinate or a derivative thereof for use in identifying a peripheral nerve, in particular a severed peripheral nerve.

A 5-aminolevulinate is a molecule that can be converted (in vivo, in vitro, or ex vivo) into PpIX. The 5-aminolevulinate or derivative thereof referred to herein may be 5-aminolevulinate (i.e. the zwitterion of 5-aminolevulinic acid), 5-aminolevulinic acid (5-ALA), a protein (e.g. peptide) derivative of a 5-aminolevulinate, an ester derivative of a 5-aminolevulinate, or a salt derivative of a 5-aminolevulinate.

The formula of 5-ALA is referred to herein as Formula I, as follows:

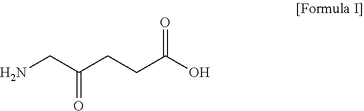

5-aminolevulinates, such as 5-ALA, are water soluble because of their zwitterionic properties. Therefore, they do not require a (toxic) vehicle, a conjugate or a specialised formulation to make them soluble. The relatively low molecular weight (MWt: 131.1 Da) of 5-ALA also enables it to penetrate the blood-nerve barrier.

The ester derivative may be one or more selected from the group consisting of 5-aminolevulinic acid methyl ester (methyl-5-aminolevulinate), 5-aminolevulinic acid ethyl ester, 5-aminolevulinic acid propyl ester, 5-aminolevulinic acid butyl ester and 5-aminolevulinic acid pentyl ester. The ester derivative may be an ester of ALA, for example, an ALA in which the carboxylic acid group has formed an ester. The ester derivative may be an N-terminus substituted ALA derivative, e.g. N-acylated derivatives.

The salt derivative may be an acid addition salt, a metal salt, an ammonium salt or an organic amine addition salt of ALA. The salt derivative may be 5-ALA·HCl. The salt derivative may be an alkyl aminolevulinate salt, such as a methyl aminolevulinate salt. The salt derivative may be a potassium salt or a sodium salt.

The protein (e.g. peptide) derivative may be N-acetyl-leucine-ALA or a N-acetyl-leucine-ALA ester derivative.

The 5-aminolevulinate derivative may be a compound produced in the porphyrin synthesis pathway, such as one or more compounds selected from the group consisting of: porphobilinogen, uroporphyrinogen I, uroporphyrinogen III coproporphyrinogen III and protoporphyrinogen IX.

The skilled person will appreciate that the 5-aminolevulinate (e.g. 5-ALA) or derivative thereof referred to herein is used as a nerve-highlighting fluorescent contrast agent.

The 5-aminolevulinate (e.g. 5-ALA) or derivative thereof may be administered systemically (e.g. orally or intravenously) or topically (e.g. eye drop). Preferably the 5-aminolevulinate (e.g. 5-ALA) or derivative thereof is administered orally.

The method according to the invention may be selective for peripheral nerves. The peripherial nerve may be a spinal nerve or a cranial nerve. The spinal nerve may be the sciatic nerve or a nerve of the brachial plexus. The cranial nerve may be a nerve that is not part of the CNS. Thus, the cranial nerve may be one or more of cranial nerves III, IV, and VI to XII, particularly the facial nerve (VII). The method according to the invention may be an in vivo method, an in vitro method, or an ex vivo method. Thus, for example, the step of contacting a nerve with a 5-aminolevulinate (e.g. 5-ALA) or a derivative thereof may occur in vivo, in vitro or ex vivo. Similarly, the step of exposing the nerve to light may occur in vivo, in vitro or ex vivo. Preferably the contacting and exposing steps occur in vivo, in vitro or ex vivo. However, the contacting and exposing steps may occur in different situations. For example, the contacting step may occur in vivo while the exposing step occurs in vitro or ex vivo.

The method according to the invention may be performed in vivo, in vitro, or ex vivo. If the method is performed in vivo, it may be performed in a subject. Thus, the step of contacting a nerve with a 5-aminolevulinate (e.g. 5-ALA) or a derivative thereof may comprise administering a 5-aminolevulinate (e.g. 5-ALA) or a derivative thereof to a subject. Thus, the invention may comprise intraoperative labelling and/or visualisation of peripheral nerves in a subject. If the method is performed in vivo, the step of performing surgery on the subject while the peripheral nerve is being visualised may comprise performing surgery on a body part of the subject. Thus the method may comprise placing a body part of the subject (on which the surgery is to be performed) in a surgical field and performing surgery on the body part of the subject while it is within the surgical field. Preferably the body part comprises the peripheral nerve. If the method is performed ex vivo, the step of contacting a nerve with a 5-aminolevulinate (e.g. 5-ALA) or a derivative thereof may comprise administering a 5-aminolevulinate (e.g. 5-ALA) or a derivative thereof to a body part that has been separated from a subject. Thus, the method may comprise placing a separated body part of a subject (on which the surgery is to be performed) in a surgical field and performing surgery on the body part. The ex vivo method may further comprise returning the body part to the subject after the step of performing surgery on the body part.

The 5-aminolevulinate or derivative thereof referred to herein may be used at a dose of about 10 mg/kg to about 50 mg/kg, 15 mg/kg to about 40 mg/kg, 15 mg/kg to about 30 mg/kg, 20 mg/kg to about 40 mg/kg, or about 20 mg/kg to about 30 mg/kg (in humans). Preferably, the 5-aminolevulinate (e.g. 5-ALA) or derivative thereof is used at a dose of about 15 mg/kg to about 30 mg/kg, or about 20 mg/kg to about 30 mg/kg (in humans). The 5-aminolevulinate or derivative thereof referred to herein may be used at a dose of about 25 mg/kg to about 200 mg/kg (in rodents, such as rats).

The 5-aminolevulinate (e.g. 5-ALA) or derivative thereof referred to herein may be a composition, such as a pharmaceutical composition comprising a 5-aminolevulinate (e.g. 5-ALA) or a derivative thereof and a pharmaceutically acceptable vehicle. The invention can be used to visualise a nerve with a diameter of about 0.1 mm or greater.

The method according to the invention may or may not be a method of surgery, a method of treatment or a method of diagnosis. The method may be non-therapeutic.

It will be appreciated that the term “surgery” as used herein can refer to treatment of disease or injury by operation or manipulation and/or an operation for exploratory or diagnostic purposes.

It will be appreciated that the term “treatment” and “treating” as used herein means the management and care of a subject for the purpose of combating a condition, such as a disease or a disorder. The term is intended to include the full spectrum of treatments for a given condition from which the subject is suffering, including alleviating symptoms or complications, delaying the progression of the disease, disorder or condition, alleviating or relieving the symptoms and complications, and/or to cure or eliminating the disease, disorder or condition as well as to prevent the condition, wherein prevention is to be understood as the management and care of a subject for the purpose of combating the disease, condition, or disorder and includes the administration of the treatment to prevent the onset of the symptoms or complications.

The “subject” is preferably a mammal, in particular a human, but it may also include animals, such as dogs, cats, horses, cows, sheep and pigs. The subject may be a body part of the subject.

“A peripheral nerve” refers to one or more nerves located in the peripheral nervous system (i.e. one or more nerves that are not part of the central nervous system). Peripheral nerves are located outside the brain and the spinal cord. A peripheral nerve typically comprises a neuron, glia and a Schwann cell. Examples of peripheral nerves include cranial nerves and spinal nerves.

A “pharmaceutically acceptable vehicle” as referred to herein, is any known compound or combination of known compounds that are known to those skilled in the art to be useful in formulating pharmaceutical compositions.

The term “visualise” can refer to “identifying”. Thus, the method of performing surgery according to the invention may comprise “visualising and identifying” a peripheral nerve.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the 5-aminolevulinate or derivative thereof) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The 5-aminolevulinate (e.g. 5-ALA) or derivative thereof referred to herein may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The 5-aminolevulinate (e.g. 5-ALA) or derivative thereof may be prepared as a sterile solid composition that may be dissolved or suspended at the time of administration using sterile water, saline, or other appropriate sterile injectable medium.

The 5-aminolevulinate (e.g. 5-ALA) or derivative thereof and the pharmaceutical compositions referred to herein may be administered orally in the form of a sterile solution or suspension containing other solutes or suspending agents (for example, enough saline or glucose to make the solution isotonic), bile salts, acacia, gelatin, sorbitan monoleate, polysorbate 8o (oleate esters of sorbitol and its anhydrides copolymerized with ethylene oxide) and the like. The 5-aminolevulinate (e.g. 5-ALA) or derivative thereof can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The term “comprising” may refer to “consisting of” or “consisting essentially of”.

All of the embodiments and features described herein (including any accompanying claims, abstract and drawings), and/or all of the steps of any method or process so disclosed, may be combined with any of the above aspects or embodiments in any combination, unless stated otherwise with reference to a specific combinations, for example, combinations where at least some of such features and/or steps are mutually exclusive.

For a better understanding of the invention, and to show embodiments of the invention may be put into effect, reference will now be made, by way of example, to the accompanying drawings, in which:—

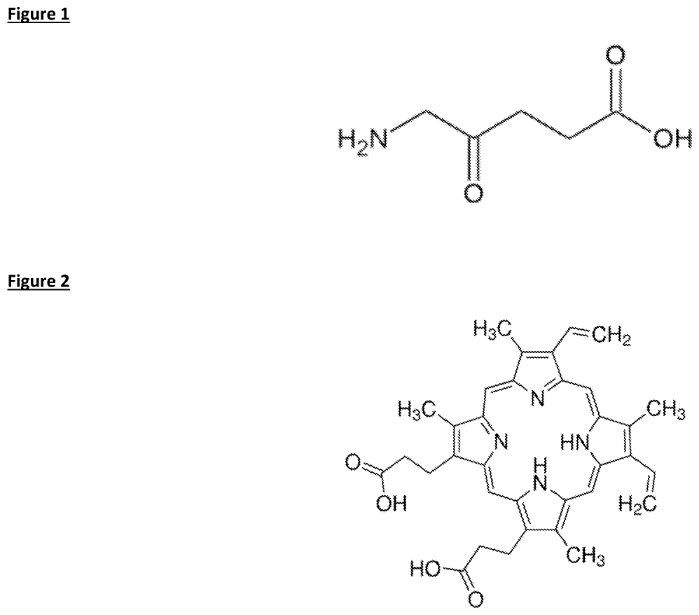

FIG. 1 shows the structure of 5-aminolevulinic acid (5-ALA), which is a delta amino acid.

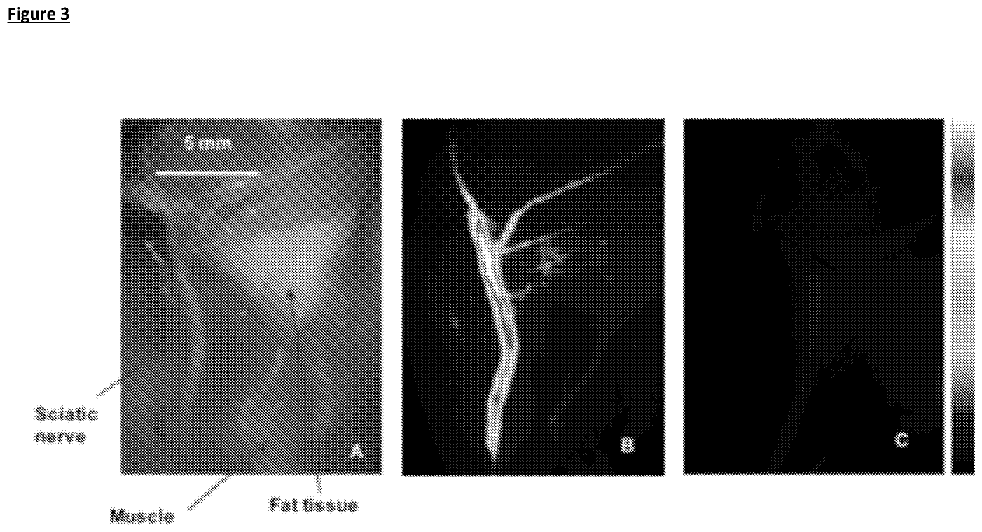

FIG. 2 shows the structure of protoporphyrin IX (PpIX).

FIG. 3 shows in vivo nerve imaging of sciatic nerve in rats injected with 5-ALA intravenously and in control rats injected only with PBS/water (without any drug). Images were obtained 24 hours after 5-ALA administration. A: bright field, B: fluorescence image with false colour scale (highest intensity red/white) in rats injected with porphyrin prodrug, C: control rat without any drug injection. Images were obtained with a CCD camera following excitation at 405 nm using a light-emitting diode (LED) and red porphyrin fluorescence was recorded at 620-650 nm. Scale bar 5 mm.

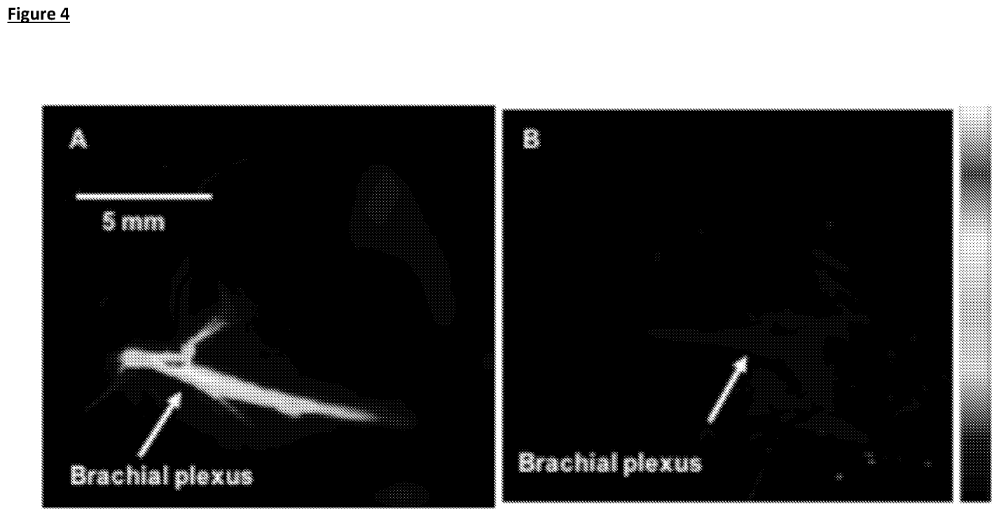

FIG. 4 shows in vivo nerve imaging of brachial plexus in rats injected with 5-ALA intravenously (A) and in control rats injected only with PBS/water (B: without any drug). Images were obtained 24 hours after 5-ALA administration using a CCD camera following excitation at 405 nm and porphyrin fluorescence was recorded at 620-650 nm. Scale bar 5 mm.

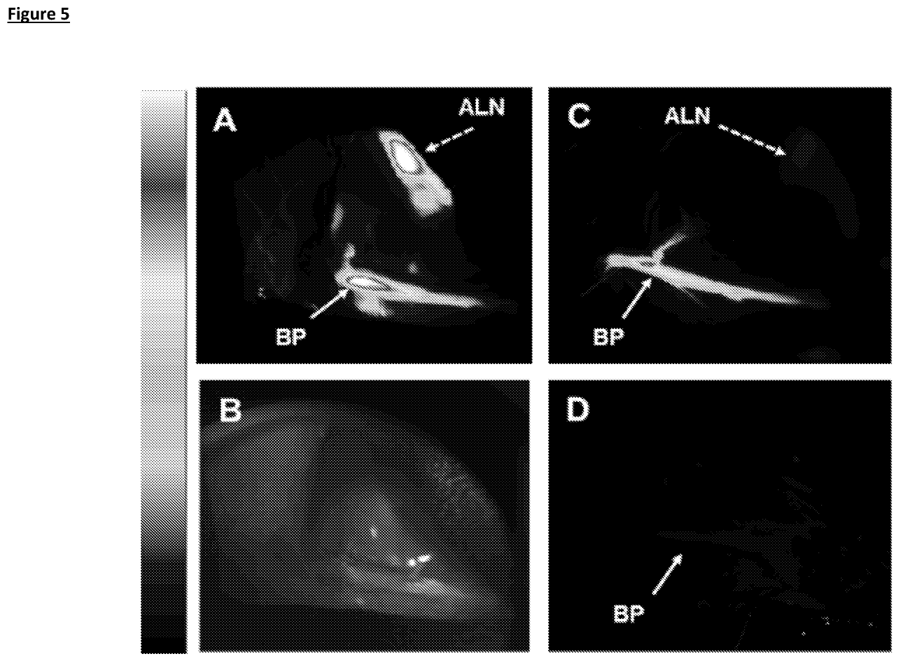

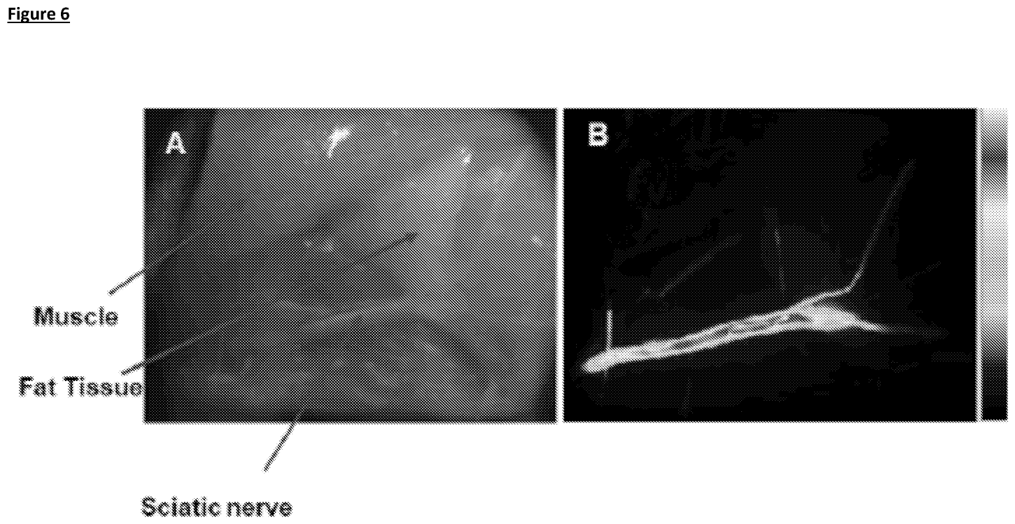

FIG. 5 shows in vivo nerve imaging of brachial plexus nerve in rats following intravenous injection of 5-ALA. False colour scale, red/white corresponding to high intensity. BP: brachial plexus, ALN: axillary lymph node. (A): Fluorescence image of the brachial plexus 4 hours post-injection, and (B) the corresponding bright field image (C): Fluorescence image of the brachial nerve 24 hours post-injection. (D): Brachial plexus image in control rat without 5-ALA injection. FIG. 4C shows the same field as FIG. 3A.

FIG. 6 shows an in vivo image of a sciatic nerve in a rat following oral administration of ALA. Images were obtained 24 hours after 5-ALA administration. (A) Bright field, (B) fluorescence image with false colour scale (highest intensity red/white) in rats administered with 5-ALA. Images were obtained with a CCD camera following excitation at 405 nm using a light-emitting diode (LED) and red porphyrin fluorescence was recorded at 620-650 nm.

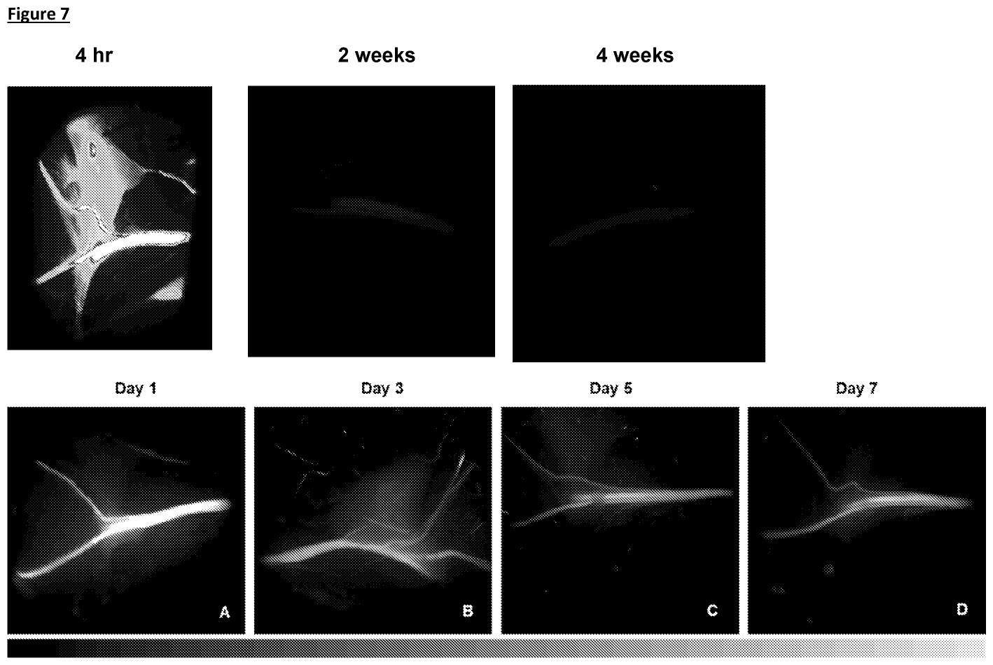

FIG. 7 shows in vivo nerve imaging of sciatic nerves in rats injected with 5-ALA intravenously at various times after intravenous administration of 5-ALA: 4 hours, day 1 (24 hours), day 3, day 5, day 7 (1 week), day 14 (2 weeks) and day 28 (4 weeks). Images were obtained 24 hours after 5-ALA administration using a CCD camera following excitation at 405 nm and porphyrin fluorescence was recorded at 620-650 nm. Scale bar 5 mm.

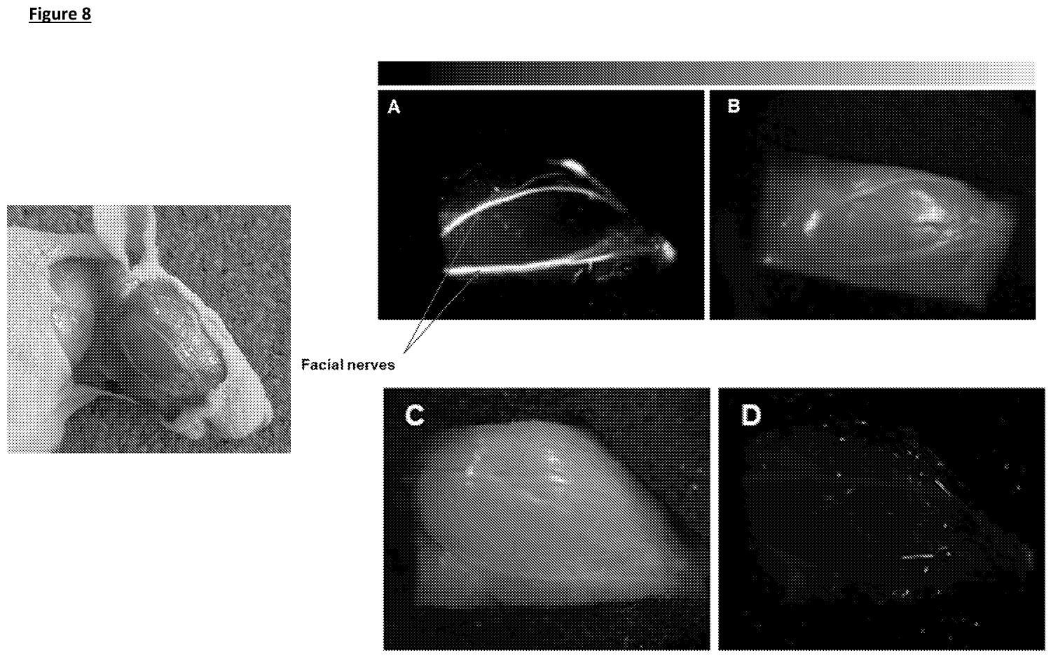

FIG. 8 shows an in vivo image of a facial nerve in a rat following intravenous administration of 5-ALA. Images were obtained 24 hours after 5-ALA administration. (A) fluorescence image (highest intensity white colour) in rats administered with 5-ALA, (B) Bright field, in rats administered with 5-ALA, (C) Bright field, in rats administered with 5-control, and (D) fluorescence image (highest intensity white colour) in rats administered with control. Images were obtained with a CCD camera following excitation at 405 nm using a light-emitting diode (LED) and red porphyrin fluorescence was recorded at 620-650 nm.

### EXAMPLES

### Materials and Methods

5-ALA was obtained from Sigma as an HCl salt in powder form, was stored inside a freezer. Before use, it was thawed, dissolved in a buffered aqueous solution at about pH 5, and then administered intravenously or orally.

### Example 1—In Vivo Peripheral Nerve Labelling in Rats Using a Porphyrin Prodrug

To investigate the potential of a porphyrin prodrug for in vivo peripheral nerve labelling, Wistar rats (n>10 were injected intravenously with the porphyrin prodrug (5-aminolevulinic acid, 5-ALA), as shown in FIG. 1. At set time-points after the injection, the rats were sacrificed and the sciatic nerves and brachial plexus (near the armpit) and surrounding tissues (e.g. muscle, fat tissues) were exposed. The fluorescence image was recorded using a CCD camera.

As shown in FIGS. 2 and 3, highly selective and localised red porphyrin fluorescence in rat nerves following intravenous injection of 5-ALA was observed. The images clearly show in vivo sciatic nerve labelling (FIG. 2) and brachial plexus labelling (FIGS. 3 and 4) in rats administered with the porphyrin prodrug. A very high fluorescence signal-to-background ratio (SBR) compare to adjacent tissues were obtained. For instance, in the sciatic nerve, the nerve to muscle intensity ratio is >15 and the nerve to fat tissue ratio is >10. This is without any optimisation (e.g. vs. dose, time) and therefore represents very promising results. Small nerve branches with a diameter of ˜0.1 mm can also be resolved. Control images elicited no contrast between muscle/nerve (FIG. 2C). Short exposures could be used (<1s) therefore video-rate imaging is feasible. The selective labelling relies on longer retention of the fluorescence in the nerve compared to surrounding tissue where the porphyrin levels reach a peak near 4 hours and then decline rapidly to basal levels in surrounding tissues.

As shown FIG. 6, highly selective and localised red porphyrin fluorescence in rat nerves following oral administration of 5-ALA was observed. A high fluorescence signal-to-background ratio (SBR) on the peripheral nerves was observed compared to adjacent tissues. The fluorescence signal ratio following oral administration was comparable to the intravenous injection route. The results were reproducible and were tested in 5 rats. Control images showed no contrast (data not shown).

### Example 2—In Vivo Visualisation of Peripheral Nerves at Various Times Following Intravenous Administration of 5-ALA

Wistar rats were administered with 5-ALA intravenously at various concentrations including 100 mg/kg and 200 mg/kg. In vivo visualisation of peripheral nerves such as sciatic nerves and brachial plexus were obtained at various post injection times ranging from 1 hours up to few weeks after administration of 5-ALA. At 4 hours FIG. 7 (A) shows bright and localised 5-ALA-induced fluorescence (protoporphyrin IX: PpIX) in sciatic nerve 24 hours after administration of 5-ALA. 5-ALA-induced fluorescence was also detected 4 hours after administration of 5-ALA. At longer time points, such as 3 days and 5 days after systemic administration of 5-ALA, sciatic nerves were also clearly visualised (FIGS. 7B and C). Fluorescence signal in nerves gradually decreased over time (FIG. 7D) indicating gradual clearance of PpIX from nerve over time. At 2 weeks and 4 weeks after administration of 5-ALA, fluorescence intensity returned close to background levels. The same results were achieved for other peripheral nerves including brachial plexus.

### Example 3—In Vivo Visualisation of Facial Nerves Following Intravenous Administration of 5-ALA

Wistar rats were administered with 5-ALA intravenously at different concentrations including 0 mg/kg (control), 100 mg/kg and 200 mg/kg. In vivo visualisation of facial nerves which are part of the cranial nerves were performed at various times including 24 hours after administration of 5-ALA. As with spinal nerves (e.g., sciatic nerves, brachial plexus), bright and localised 5-ALA induced fluorescence (protoporphyrin IX: PpIX) was detected in various branches of facial nerves (FIG. 8A). No fluorescence was detected in control rats (FIG. 8D).

These results confirm that with 5-ALA both the spinal nerves and cranial nerves can be detected and visualised.

1. A method of visualising a peripheral nerve, the method comprising:
contacting the nerve with a 5-aminolevulinate or a derivative thereof;
exposing the nerve to light at least about 1 hour after contact with the 5-aminolevulinate or derivative thereof in order to cause the peripheral nerve to emit fluorescence; and
detecting the fluorescence emitted by the nerve to visualise the peripheral nerve.

2. A method of fluorescently labelling a peripheral nerve, the method comprising: contacting the peripheral nerve with a 5-aminolevulinate or derivative thereof at least about 1 hour prior to exposing the nerve to light.

3. A method of performing surgery on a subject, the method comprising:
administering a 5-aminolevulinate or a derivative thereof to a subject;
exposing a peripheral nerve of the subject to light at least about 1 hour after administration of 5-aminolevulinate or derivative thereof, in order to cause the peripheral nerve to emit fluorescence;
detecting the fluorescence emitted by the peripheral nerve of the subject in order to visualise the peripheral nerve; and
performing surgery on the subject while the peripheral nerve is being visualised.

4. A 5-aminolevulinate or a derivative thereof for use in (a method of) surgery on a subject.

5. The 5-aminolevulinate or a derivative thereof for use according to claim 4, comprising:
administering the 5-aminolevulinate or a derivative thereof to the subject;
exposing peripheral nerves of the subject to light at least about 1 hour after administration of 5-aminolevulinate or derivative thereof, in order to cause the peripheral nerve to emit fluorescence;
detecting the fluorescence emitted by the peripheral nerve of the subject in order to visualise the peripheral nerve; and
performing surgery on the subject while the peripheral nerve is being visualised.

6. The method of claim 3, or the 5-aminolevulinate or derivative thereof for use according to claim 5, wherein the step of administering is topical administration of 5-aminolevulinate or a derivative thereof, or systemic administration of 5-aminolevulinate or a derivative thereof, such oral administration.

7. The method of claim 6, or the 5-aminolevulinate or derivative thereof for use according to claim 6, wherein systemic administration is oral and/or intravenous administration of 5-aminolevulinate or a derivative thereof.

8. The method of any one of claims 1, 3, 6 or 7, or the 5-aminolevulinate or derivative thereof for use according to any one of claims 5 to 7, wherein the step of exposing the peripheral nerve to light comprises exposing the peripheral nerve to red light (about 600 nm to about 700 nm) or blue light (about 380 nm to about 495 nm).

9. The method of any one of claims 1, 3, 6, 7 or 8, or the 5-aminolevulinate or derivative thereof for use according to any one of claims 5 to 8, wherein the peripheral nerve is exposed to light with a wavelength of about 620 nm to about 650 nm.

10. The method of any one of claims 1, 3, 6, 7, 8 or 9, or the 5-aminolevulinate or derivative thereof for use according to any one of claims 5 to 9, comprising exposing the peripheral nerve to light about 4 hours to about 48 hours or about 20 hours to about 30 hours after the administration/contact step.

11. The method of any one of claims 1, 3, 6, 7, 8, 9 or 10, or the 5-aminolevulinate or derivative thereof for use according to any one of claims 5 to 10, wherein the step of detecting fluorescence comprises detecting fluorescence emitted at about 620 nm to about at about 650 nm at least 1 hour after the nerve has been exposed to blue light.

12. The method of any one of claims 3, 6, 7, 8, 9, 10 or 11, or the 5-aminolevulinate or derivative thereof for use according to any one of claims 5 to 11, wherein the step of performing surgery on the subject in view of the peripheral nerve is performed in constant view of the peripheral nerve or with intermittent view of the peripheral nerve.

13. The 5-aminolevulinate or derivative thereof for use according to claim 11 or claim 12, wherein the surgery is one or more selected from the group comprising cancer, non-cancer surgery, head and neck surgery, a mastectomy, thoracic surgery, prostatectomy, cardiothoracic surgery (e.g. coronary artery bypass), gynaecological surgery, colorectal surgery, and trauma and orthopaedic surgery.

14. The method of any one of claims 1 to 3 or 6 to 12, or the 5-aminolevulinate or derivative thereof for use according to any one of claims 5 to 13, wherein the 5-aminolevulinate is 5-aminolevulinic acid (5-ALA).

15. The method according to claim 14, or the 5-aminolevulinate or a derivative thereof for use according to any one of claim 14, wherein 5-aminolevulinic acid has the formula (referred to herein as Formula I):

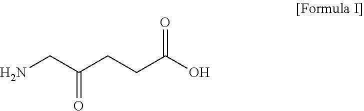

16. The method according to claim 14 or claim 15, or the 5-aminolevulinate or a derivative thereof for use according to claim 14 or claim 15, wherein the derivative thereof is a protein (e.g. peptide) derivative thereof, an ester derivative thereof or a salt derivative thereof.
